(19) United States
(12) Patent Application Publication
(10) Pub. No.: US 2022/0241170 A1
(43) Pub. Date: Aug. 4, 2022

(54) EYELASH COMPOSITIONS CONTAINING SUGAR

(71) Applicant: L'OREAL, Paris (FR)

(72) Inventors: Christopher PANG, Nanuet, NY (US); Tianyi LIU, Springfield, PA (US)

(73) Assignee: L'OREAL, Paris (FR)

(21) Appl. No.: 17/160,750

(22) Filed: Jan. 28, 2021

(51) Int. Cl. A61K 8/60; A61Q 1/10

(52) U.S. Cl. CPC A61K 8/60; A61K 2800/31; A61K 2800/43; A61Q 1/10

(57) ABSTRACT

The invention relates to an eyelash composition, especially a mascara composition, including more than 5% by weight of the eyelash composition of at least one sugar, as well as to methods of using such compositions.

### FIELD OF THE INVENTION

The present invention relates to eyelash compositions comprising at least one sugar. Among other improved or beneficial properties, eyelash compositions containing at least one sugar have surprisingly good removal properties.

### DISCUSSION OF THE BACKGROUND

Eyelash compositions such as mascaras are typically two types of formulations: aqueous mascaras generally referred to as cream mascaras, generally in the form of an emulsion of waxes in water, and anhydrous or low-water-content mascaras, generally referred to as water-resistant (also referred to as “waterproof”), generally in the form of dispersions of waxes in organic solvents.

Aqueous compositions generally contain a surfactant system, for example based on triethanolamine stearate, which makes it possible to obtain a stable dispersion of particles of wax agglomerated in an aqueous phase. This system plays an important part in obtaining a stable dispersion, in particular at the interface in the interactions between particles of wax.

Anhydrous compositions can have the disadvantages of, among other things, being dry, having poor flexibility, poor consistency and/or high viscosities that can produce lumps on the lashes. They also are generally difficult to remove from eyelashes.

There remains a need for improved eyelash compositions having good cosmetic properties which can be easily removed from eyelashes (“removability”).

Accordingly, one aspect of the present invention is a care and/or makeup and/or treatment composition for eyelashes which has good cosmetic properties and which has good removability.

### SUMMARY OF THE INVENTION

The present invention relates to low-water-content eyelash compositions comprising at least one sugar and at least one oil. Preferably, the compositions are anhydrous.

The present invention also relates to low-water-content colored eyelash compositions comprising at least one coloring agent, at least one oil, and at least one sugar. Such compositions can be mascara compositions. Preferably, the low-water-content colored eyelash compositions are anhydrous.

The present invention also relates to methods of treating, caring for and/or making up eyelashes by applying compositions of the present invention comprising at least one sugar and at least one oil to the eyelashes in an amount sufficient to treat, care for and/or make up the eyelashes.

The present invention also relates to methods of enhancing the appearance of eyelashes by applying compositions of the present invention comprising at least one sugar and at least one oil to the eyelashes in an amount sufficient to enhance the appearance of the eyelashes.

The present invention also relates to methods of removing a low-water-content eyelash composition from eyelashes, wherein the eyelash composition comprises at least one sugar in an amount greater than 5% by weight of the eyelash composition and at least one oil, comprising applying water to the eyelash composition in an amount sufficient to remove the eyelash composition from the eyelashes.

The present invention also relates to methods of increasing removability of a low-water-content eyelash composition from eyelashes comprising adding during preparation of the eyelash composition at least one sugar in an amount greater than 5% by weight of the eyelash composition to the eyelash composition, resulting in increased removability of the eyelash composition from eyelashes.

It is to be understood that both the foregoing general description and the following detailed description are exemplary and explanatory only, and are not restrictive of the invention.

### DETAILED DESCRIPTION OF THE INVENTION

As used herein, the expression “at least one” means one or more and thus includes individual components as well as mixtures/combinations.

Other than in the operating examples, or where otherwise indicated, all numbers expressing quantities of ingredients and/or reaction conditions are to be understood as being modified in all instances by the term “about,” meaning within 10% of the indicated number.

“Film former” or “film forming agent” as used herein means a polymer or resin that leaves a film on the substrate to which it is applied, for example, after a solvent accompanying the film former has evaporated, absorbed into and/or dissipated on the substrate.

“Transfer resistance” as used herein refers to the quality exhibited by compositions that are not readily removed by contact with another material, such as, for example, an item of clothing or the skin. Transfer resistance may be evaluated by any method known in the art for evaluating such. For example, transfer resistance of a composition may be evaluated by a modified “kiss” test or material transfer test. This test may involve application of the composition to human keratin material such as eyelashes followed by rubbing a material, for example, a sheet of paper or cotton ball, against the eyebrows after expiration of a certain amount of time following application, such as 2 minutes after application. Similarly, transfer resistance of a composition may be evaluated by the amount of product transferred from a wearer to any other substrate, such as transfer from the eyelashes of an individual to an article of clothing when putting on the clothing after the expiration of a certain amount of time following application of the composition to the eyelashes. The amount of composition transferred to the substrate (e.g., clothing, paper or cotton ball) may then be evaluated and compared. For example, a composition may be transfer resistant if a majority of the product is left on the wearer's eyelashes. Further, the amount transferred may be compared with that transferred by other compositions, such as commercially available compositions. In a preferred embodiment of the present invention, little or no composition is transferred to the substrate from the eyelashes.

“Long wear” compositions as used herein, refers to compositions where color remains the same or substantially the same as at the time of application, as viewed by the naked eye, after an extended period of time. Long wear properties may be evaluated by any method known in the art for evaluating such properties. For example, long wear may be evaluated by a test involving the application of a composition to eyelashes and evaluating the color of the composition after an extended period of time. For example, the color of a composition may be evaluated immediately following application to eyelashes and these characteristics may then be re-evaluated and compared after a certain amount of time such as after 24 hours or 36 hours. Further, these characteristics may be evaluated with respect to other compositions, such as commercially available compositions.

“Waterproof” properties as used herein may be evaluated by any method known in the art for evaluating such properties. For example, such properties may be evaluated by applying a composition to eyelashes and evaluating the color of the composition after an extended period of time and exposure to water. For example, the color of a composition may be evaluated immediately following application to eyelashes and these characteristics may then be re-evaluated and compared after a certain amount of time such as after 20 minutes. Also for example, application of water can occur through various means including exposure to sauna conditions (40-45% humidity and temperatures of 50° C.-56° C. or pool test (humidity 56.6-87.1%) for a defined period of time (for example, 20 minutes). Further, these characteristics may be evaluated with respect to other compositions, such as commercially available compositions using the same known methodologies.

“Substituted” as used herein, means comprising at least one substituent. Non-limiting examples of substituents include atoms, such as oxygen atoms and nitrogen atoms, as well as functional groups, such as hydroxyl groups, ether groups, alkoxy groups, acyloxyalky groups, oxyalkylene groups, polyoxyalkylene groups, carboxylic acid groups, amine groups, acylamino groups, amide groups, halogen containing groups, ester groups, thiol groups, sulphonate groups, thiosulphate groups, siloxane groups, and polysiloxane groups. The substituent(s) may be further substituted.

“Volatile”, as used herein, means having a flash point of less than about 100° C.

“Non-volatile”, as used herein, means having a flash point of greater than about 100° C.

“Low-water-content” means the compositions contain less than 6% water (by weight). Preferably, compositions of the present invention comprising the at least one sugar contain less than 5% water, and preferably less than 4% water.

“Anhydrous” means the compositions contain less than 1% water (by weight). Preferably, anydrous compositions of the present invention comprising the at least one sugar contain less than 0.5% water, and preferably no water.

“Polymer” as used herein means a compound which is made up of at least two monomers.

“Free” or “substantially free” or “devoid of” as it is used herein means that while it is preferred that no amount of the specific component be present in the composition, it is possible to have very small amounts of it in the compositions of the invention provided that these amounts do not materially affect at least one, preferably most, of the advantageous properties of the conditioning compositions of the invention. Thus, for example, “free of triethanolamine (TEA)” means that TEA is omitted from the composition (that is, 0% by weight), “substantially free of TEA” means that TEA is are present in amounts not greater than 0.5% by weight, and “devoid of TEA” means that TEA is present in amounts not greater than 0.25% by weight, based on the total weight of the composition. The same nomenclature applies for all other ingredients identified throughout the application such as, for example, silicone oils (“free of silicone oils,” “substantially free of silicone oils,” and “devoid of silicone oils” have meanings consistent with the discussion within this paragraph), even if not specifically discussed for each identified ingredient. Discussed examples of the use of such language are intended to be exemplary, not limiting.

The compositions and methods of the present invention can “comprise,” “consist of” or “consist essentially of” the essential elements and limitations of the invention described herein, as well as any additional or optional ingredients, components, or limitations described herein or otherwise useful. For purposes of the compositions and methods of the present invention where the invention “consists essentially of” the identified ingredients and/or process steps, the “basic and novel property” of such compositions and/or methods is “removability” from eyelashes.

Sugar

According to the present invention, compositions comprising at least one sugar are provided.

“Sugar” as used herein refers to solid, water-soluble, and oil-insoluble compounds which have one or more alcohol functional groups, with or without an aldehyde or ketone functional group, and which include at least 4 carbon atoms and preferably include from 5 to 6 carbon atoms. Examples of sugars which can be used according to the invention are sugars belonging to the groups consisting of the oses or monosaccharides and their derivatives such as the polyols, and/or to the groups consisting of the holosides, in particular the diholosides. Suitable sugars include, for example, sucrose, glucose, galactose, ribose, fucose, trehalose, maltose, fructose, mannose, arabinose, xylose, lactose, their derivatives, in particular their alkylated derivatives, such as methylated derivatives, for example methylglucose, as well as compounds that contain one or more sugars, and their mixtures.

According to the present invention, the at least one sugar is present in the compositions of the present invention in an amount greater than 5% by weight of the composition, preferably greater than about 7.5% by weight of the composition, preferably greater than about 10% by weight of the composition, preferably greater than about 15% by weight of the composition, and preferably greater than about 20% by weight of the composition, and preferably the at least one sugar is present in the compositions of the present invention in an amount of less than 50% by weight of the composition, preferably less than about 40% by weight of the composition, preferably less than about 30% by weight of the composition, and preferably less than about 25% by weight of the composition, including all ranges and subranges therebetween such as, for example, 7.5% to 40%, 7.5% to 25%, 10% to 30%, 20% to 30%, etc.

Oil

According to the present invention, compositions comprising at least one oil are provided. “Oil” means any non-aqueous medium which is liquid at ambient temperature (25° C.) and atmospheric pressure (760 mm Hg).

Suitable oils include volatile and/or non-volatile oils. Such oils can be any acceptable oil including but not limited to silicone oils and/or hydrocarbon oils.

According to certain embodiments, the compositions of the present invention preferably comprise one or more volatile silicone oils. Examples of such volatile silicone oils include linear or cyclic silicone oils having a viscosity at room temperature less than or equal to 6 cSt and having from 2 to 7 silicon atoms, these silicones being optionally substituted with alkyl or alkoxy groups of 1 to 10 carbon atoms. Specific oils that may be used in the invention include octamethyltetrasiloxane, decamethylcyclopentasiloxane, dodecamethylcyclohexasiloxane, heptamethyloctyltrisiloxane, hexamethyldisiloxane, decamethyltetrasiloxane, dodecamethylpentasiloxane and their mixtures. Other volatile oils which may be used include KF 96A of 6 cSt viscosity, a commercial product from Shin Etsu having a flash point of 94° C. Preferably, the volatile silicone oils have a flash point of at least 40° C.

Non-limiting examples of volatile silicone oils are listed in Table 1 below.

TABLE 1

| | Flash Point | Viscosity |
|---|---|---|
| Compound | (° C.) | (cSt) |
| Octyltrimethicone | 93 | 1.2 |
| Hexyltrimethicone | 79 | 1.2 |
| Decamethylcyclopentasiloxane | 72 | 4.2 |
| (cyclopentasiloxane or D5) | | |
| Octamethylcyclotetrasiloxane | 55 | 2.5 |
| (cyclotetradimethylsiloxane or D4) | | |
| Dodecamethylcyclohexasiloxane (D6) | 93 | 7 |
| Decamethyltetrasiloxane(L4) | 63 | 1.7 |
| KF-96 A from Shin Etsu | 94 | 6 |
| PDMS (polydimethylsiloxane) DC 200 | 56 | 1.5 |
| (1.5 cSt) from Dow Corning | | |
| PDMS DC 200 (2 cSt) from Dow Corning | 87 | 2 |

Further, a volatile linear silicone oil may be employed in the present invention. Suitable volatile linear silicone oils include those described in U.S. Pat. No. 6,338,839 and WO03/042221, the contents of which are incorporated herein by reference. In one embodiment the volatile linear silicone oil is decamethyltetrasiloxane. In another embodiment, the decamethyltetrasiloxane is further combined with another solvent that is more volatile than decamethyltetrasiloxane.

According to other embodiments, the compositions of the present invention preferably comprise one or more non-silicone volatile oils and may be selected from volatile hydrocarbon oils, volatile esters and volatile ethers. Examples of such volatile non-silicone oils include, but are not limited to, volatile hydrocarbon oils having from 8 to 16 carbon atoms and their mixtures and in particular branched C₈ to C₁₆ alkanes such as C₈ to C₁₆ isoalkanes (also known as isoparaffins), isododecane, isodecane, and for example, the oils sold under the trade names of Isopar or Permethyl. Preferably, the volatile non-silicone oils have a flash point of at least 40° C.

Non-limiting examples of volatile non-silicone volatile oils are given in Table 2 below.

TABLE 2

| Compound | Flash Point (° C.) |
|---|---|
| Isododecane | 43 |
| Propylene glycol n-butyl ether | 60 |
| Ethyl 3-ethoxypropionate | 58 |
| Propylene glycol methylether acetate | 46 |
| Isopar L (isoparaffin C₁₁-C₁₃) | 62 |
| Isopar H (isoparaffin C₁₁-C₁₂) | 56 |

The volatility of the solvents/oils can be determined using the evaporation speed as set forth in U.S. Pat. No. 6,338,839, the contents of which are incorporated by reference herein.

According to other embodiments of the present invention, the compositions of the present invention comprise at least one non-volatile oil. Examples of non-volatile oils that may be used in the present invention include, but are not limited to, polar oils such as:
hydrocarbon-based plant oils with a high triglyceride content consisting of fatty acid esters of glycerol, the fatty acids of which may have varied chain lengths, these chains possibly being linear or branched, and saturated or unsaturated; these oils are especially wheat germ oil, corn oil, sunflower oil, karite butter, castor oil, sweet almond oil, macadamia oil, apricot oil, soybean oil, rapeseed oil, cottonseed oil, alfalfa oil, poppy oil, pumpkin oil, sesame seed oil, marrow oil, avocado oil, hazelnut oil, grape seed oil, blackcurrant seed oil, evening primrose oil, millet oil, barley oil, quinoa oil, olive oil, rye oil, safflower oil, candlenut oil, passion flower oil or musk rose oil; or caprylic/capric acid triglycerides, for instance those sold by the company Stearineries Dubois or those sold under the names Miglyol 810, 812 and 818 by the company Dynamit Nobel;
synthetic oils or esters of formula R₅COOR₆ in which R₅ represents a linear or branched higher fatty acid residue containing from 1 to 40 carbon atoms, including from 7 to 19 carbon atoms, and R₆ represents a branched hydrocarbon-based chain containing from 1 to 40 carbon atoms, including from 3 to 20 carbon atoms, with R₆+R₇≥10, such as, for example, Purcellin oil (cetostearyl octanoate), isononyl isononanoate, C₁₂ to C₁₅ alkyl benzoate, isopropyl myristate, 2-ethylhexyl palmitate, and octanoates, decanoates or ricinoleates of alcohols or of polyalcohols; hydroxylated esters, for instance isostearyl lactate or diisostearyl malate; and pentaerythritol esters;
synthetic ethers containing from 10 to 40 carbon atoms;
C₈ to C₂₆ fatty alcohols, for instance oleyl alcohol, cetyl alcohol, stearyl alcohol, and cetearly alcohol; and
mixtures thereof.

Further, examples of non-volatile oils that may be used in the present invention include, but are not limited to, non-polar oils such as branched and unbranched hydrocarbons and hydrocarbon waxes including polyolefins, in particular Vaseline (petrolatum), paraffin oil, squalane, squalene, hydrogenated polyisobutene, hydrogenated polydecene, polybutene, mineral oil, pentahydrosqualene, and mixtures thereof.

According to preferred embodiments of the present invention, the oil is a high viscosity oil which is a silicone oil and/or a hydrocarbon oil. “High viscosity” means an oil having a viscosity greater than 250 cSt at 25° C.

Suitable examples of such silicone oils include, but are not limited to, non-volatile silicone fluids such as, for example, polyalkyl (aryl) siloxanes. Suitable polyalkyl siloxanes include, but are not limited to, polydimethyl siloxanes, which have the CTFA designation dimethicone, polydiethyl siloxane, phenyl trimethicone, trimethyl pentaphenyl trisiloxane, phenyldimethicone, phenyltrimethylsiloxydiphenylsiloxane, diphenyldimethicone, and diphenylmethyldiphenyltrisiloxane and those siloxanes disclosed in U.S. patent application publication no. 2004/0126350, the entire disclosure of which is hereby incorporated by reference. Specific examples of suitable high viscosity silicone oils include, but are not limited to, 15 M 30 from PCR (500 cSt) or Belsil PDM 1000 (1 000 cSt) from Wacker and Dow Corning 200 (350 cSt) (the values in parenthesis represent viscosities at 25° C.).

Suitable examples of such hydrocarbon oils include, but are not limited to, fluids having a molecular mass of more than 500 g/mol, for example more than 600 g/mol, and for example more than 650 g/mol. By “hydrocarbon” compound, it is meant a compound comprising principally atoms of carbon and hydrogen and optionally one or more functional groups chosen from hydroxyl, ester, ether and carboxyl functions. These compounds are, according to one aspect, devoid of —Si—O— groups. Suitable examples of hydrocarbon fluids include, but are not limited to polybutylenes, such as Indopol H-100 (of molar mass or MM=965 g/mol), Indopol H-300 (MM=1 340 g/mol), and Indopol H-1500 (MM=2 160 g/mol), which are sold or manufactured by Amoco; hydrogenated polyisobutylenes, such as Panalane H-300 E, sold or manufactured by Amoco (M=1 340 g/mol), Viseal 20000 sold or manufactured by Synteal (MM=6 000 g/mol), and Rewopal PIB 1000, sold or manufactured by Witco (MM=1 000 g/mol); polydecenes and hydrogenated polydecenes, such as Puresyn 10 (MM=723 g/mol) and Puresyn 150 (MM=9 200 g/mol) sold or manufactured by Mobil Chemicals; esters such as linear fatty acid esters having a total carbon number ranging from 30 to 70, such as pentaerythrityl tetrapelargonate (MM=697.05 g/mol); hydroxy esters, such as diisostearyl malate (MM=639 g/mol); aromatic esters such as tridecyl trimellitate (MM=757.19 g/mol); esters of C24-C28 branched fatty acids or fatty alcohols, such as those described in EP-A-0 955 039, for example triisocetyl citrate (MM=856 g/mol), pentaerythrityl tetraisononanoate (MM=697.05 g/mol), glyceryl triisostearate (MM=891.51 g/mol), glyceryl 2-tridecyltetradecanoate (MM=1 143.98 g/mol), pentaerythrityl tetraisostearate (MM=1 202.02 g/mol), poly-2-glyceryl tetraisostearate (MM=1 232.04 g/mol) and pentaerythrityl 2-tetradecyltetradecanoate (MM=1 538.66 g/mol); and mixtures thereof. Suitable ester oils can also be described according to formula R₁COOR₂ in which R₁ represents a linear or branched higher fatty acid residue containing from 1 to 40 carbon atoms, including from 7 to 19 carbon atoms, and R₂ represents a branched hydrocarbon-based chain containing from 1 to 40 carbon atoms, including from 3 to 20 carbon atoms, with R₁+R₂ 10, such as, for example, Purcellin oil (cetostearyl octanoate), isononyl isononanoate, C₁₂ to C₁₅ alkyl benzoate, isopropyl myristate, 2-ethylhexyl palmitate, and octanoates, decanoates or ricinoleates of alcohols or of polyalcohols; hydroxylated esters, for instance isostearyl lactate or diisostearyl malate; and pentaerythritol esters. A particularly preferred ester is diisostearyl malate.

According to preferred embodiments, the at least one oil is present in the compositions of the present invention in an amount ranging from about 10 to about 65% by weight, preferably from about 20 to about 60% by weight, and preferably from about 30 to about 55% by weight, based on the total weight of the composition, including all ranges and subranges within these ranges.

According to preferred embodiments, more oil is present in the compositions of the present invention than sugar. Preferably, the oils(s) and the sugar(s) are present in the compositions of the present invention in a weight ratio of 10:1 to 1.1:1, preferably 5:1 to 1.3:1, and preferably 3:1 to 1.5:1, including all ranges and subranges therebetween.

Wax

According to preferred embodiments of the present invention, compositions further comprising at least one wax are provided. “Wax” means a lipophilic compound, solid at ambient temperature (25° C.), with a reversible solid/liquid change of state, having a melting point greater than or equal to 30° C. Examples of suitable waxes include soft waxes and hard waxes.

“Soft waxes” may be defined as those waxes which have a melting point of below 70° C., and preferably, a melting point of below about 60° C. “Hard waxes” may be defined as those waxes which have a melting point of equal to or greater than about 70° C., and preferably, a melting point of equal to or greater than about 80° C.

Hard waxes can be hydrocarbon, fluorinated and/or silicone, and be of plant, mineral, animal and/or synthetic origin. Suitable examples of hard wax include, but are not limited to, carnauba wax, microcrystalline wax, polyethylene wax, hydrogenated castor oil, wax AC 540, Hydroxyoctacosanyl Hydroxystearate, hydrogenated castor wax, wax AC 400, rice bran wax, C20-40 alkyl stearate, Alcohol polyethylene wax, octanedioate, sunflower seed wax, fischer-tropsch wax, Chinese insect wax, shellac wax, benehyl fumarate, synthetic wax, betsawax RX-13750, phytowax ricin 22 L 73, and vegetable wax.

Soft waxes can be hydrocarbon, fluorinated and/or silicone, and be of plant, mineral, animal and/or synthetic origin. Suitable examples of soft wax include, but are not limited to, paraffin wax, stearic alcohol, ozokerite, synthetic beeswax, beeswax, candelilla wax, PVP/eicosene copolymer, hydrogenated jojoba wax, palm butter, sumac wax, polyglyceryl beeswax, tricontanyl/PVP, siliconyl beeswax, stearyl stearate, ceresin wax, hydrogenated myristyl olive esters (e.g., phytowax olive 14 L 48), hydrogenated stearyl olive esters (e.g., phytowax olive 18 L 57), Koster K82P, orange peel wax, Pentaerythritol distearate, Theobroma Grandiflorum Seed Butter, silicone resin wax, Polymethylalkyl dimethylsiloxane, Pentaerythrityl tetrastearate, Tetracontanyl Stearate, fatty acid wax, behenyl alcohol, alkyl dimethicone wax, Stearyl Benzoate, Berry wax, koster wax, siliconyl candelilla wax, Ditrimethylolpropane tetrastearate, Clariant Licowax KST 1, Dipentaerythrytol hexastearate, Ditrimethylolpropane tetrabehenate, Behenyl methacrylate greffe PDMS, jojoba esters, waxolive, inholive, phytowax ricin 16 L 64, hydrogenated macadamia seed oil, synthetic wax, dooctadecyl carbonate, montan wax, lemon peel extract, ditrimethyloylpropane tetrastearate, C30-45 alkyldimethylsilyl propylsilsesquioxane, Hest 2T-5E-45, Ditrimethylolpropane tetralaurate, Koster BK-34, Fluoro Polymethylalkyl dimethylsiloxane, Blend of Dilauryl Adipate and Ditetradecyl Adipate, Astrocaryum MuruMuru Seed Butter, Myrica Pubescens Wax, PEG-70 Mango Glycerides, oxypropylenated lanolin wax, and hydrogenated Coco-glycerides.

The wax(es) are preferably present in a quantity of from about 5% to about 35% by weight, preferably from about 10% to about 30% by weight, and preferably from about 15% to about 25% by weight relative to the total weight of the composition, including all ranges and subranges therebetween.

Coloring Agents

According to preferred embodiments of the present invention, compositions further comprising at least one coloring agent are provided. Preferably, compositions comprising at least one coloring agent are mascara compositions. In contrast, uncolored compositions of the present invention (either because they do not contain coloring agents or because they contain an amount of coloring insufficient to effectively provide color) can be compositions for treating or caring for eyelashes, or can be topcoat or basecoat compositions used in conjunction with mascara compositions.

According to this embodiment, the at least one coloring agent is preferably chosen from pigments, dyes, such as liposoluble dyes, nacreous pigments, and pearling agents.

Representative liposoluble dyes which may be used according to the present invention include Sudan Red, DC Red 17, DC Green 6, β-carotene, soybean oil, Sudan Brown, DC Yellow 11, DC Violet 2, DC Orange 5, annatto, and quinoline yellow. The liposoluble dyes, when present, generally have a concentration ranging up to 20% by weight of the total weight of the composition, such as from 0.0001% to 6%, including all ranges and subranges therebetween.

The nacreous pigments which may be used according to the present invention may be chosen from white nacreous pigments such as mica coated with titanium or with bismuth oxychloride, colored nacreous pigments such as titanium mica with iron oxides, titanium mica with ferric blue or chromium oxide, titanium mica with an organic pigment chosen from those mentioned above, and nacreous pigments based on bismuth oxychloride. The nacreous pigments, if present, be present in the composition in a concentration ranging up to 50% by weight of the total weight of the composition, such as from 0.1% to 20%, preferably from 0.1% to 15%, including all ranges and subranges therebetween.

The pigments, which may be used according to the present invention, may be chosen from white, colored, inorganic, organic, polymeric, nonpolymeric, coated and uncoated pigments. Representative examples of mineral pigments include titanium dioxide, optionally surface-treated, zirconium oxide, zinc oxide, cerium oxide, iron oxides, chromium oxides, manganese violet, ultramarine blue, chromium hydrate, and ferric blue. Representative examples of organic pigments include carbon black, pigments of D & C type, and lakes based on cochineal carmine, barium, strontium, calcium, and aluminum. If present, the pigments may be present in the composition in a concentration ranging up to 50% by weight of the total weight of the composition, such as from 0.5% to 40%, and further such as from 2% to 30%, including all ranges and subranges therebetween. In the case of certain products, the pigments, including nacreous pigments, may, for example, represent up to 50% by weight of the composition.

Additional Additives

The composition of the invention can also comprise any additive usually used in the field under consideration. For example, dispersants, clays, antioxidants, sunscreens, preserving agents, fragrances, fibers, fillers, neutralizing agents, cosmetic and dermatological active agents such as, for example, emollients, moisturizers, vitamins, essential fatty acids, surfactants, silicone elastomers, pasty compounds, viscosity increasing agents, film forming agents, and mixtures thereof can be added. A non-exhaustive listing of such ingredients can be found in U.S. patent application publication nos. 2004/0170586 and 2009/0142289, the entire contents of which are hereby incorporated by reference. Further examples of suitable additional components can be found in the other references which have been incorporated by reference in this application. Still further examples of such additional ingredients may be found in the International Cosmetic Ingredient Dictionary and Handbook (9^{th} ed. 2002).

Suitable clays include, but are not limited to, lipophilic or hydrophilic clays. The term “hydrophilic clay” means a clay that is capable of swelling in water; this clay swells in water and forms after hydration a colloidal dispersion. These clays are products that are already well known per se, which are described, for example, in the book “Mineralogie des argiles”, S. Caillere, S. Henin, M. Rautureau, 2^{nd} edition 1982, Masson, the teaching of which is included herein by way of reference. Clays are silicates containing a cation that may be chosen from calcium, magnesium, aluminium, sodium, potassium and lithium cations, and mixtures thereof. Examples of such products that may be mentioned include clays of the smectite family such as montmorillonites, hectorites, bentonites, beidellites and saponites, and also of the family of vermiculites, stevensite and chlorites. These clays may be of natural or synthetic origin.

Hydrophilic clays that may be mentioned include smectite products such as saponites, hectorites, montmorillonites, bentonites and beidellite. Hydrophilic clays that may be mentioned include synthetic hectorites (also known as laponites), for instance the products sold by the company Laporte under the names Laponite XLG, Laponite RD and Laponite RDS (these products are sodium magnesium silicates and in particular sodium lithium magnesium silicates); bentonites, for instance the product sold under the name Bentone HC by the company Rheox; magnesium aluminium silicates, especially hydrated, for instance the products sold by the Vanderbilt Company under the names Veegum Ultra, Veegum HS and Veegum DGT, or calcium silicates, and especially the product in synthetic form sold by the company under the name Micro-cel C.

The term “lipophilic clay” means a clay that is capable of swelling in a lipophilic medium; this clay swells in the medium and thus forms a colloidal dispersion. Examples of lipophilic clays that may be mentioned include modified clays such as modified magnesium silicate (Bentone Gel VS38 from Rheox), and hectorites modified with a C₁₀ to C₂₂ fatty-acid ammonium chloride, for instance hectorite modified with distearyldimethylammonium chloride (CTFA name: disteardimonium hectorite) sold under the name Bentone 38 CE by the company Rheox or Bentone 38V® by the company Elementis.

Preferably, the clay(s), if present, are present in the composition of the present invention in amounts of active material generally ranging from about 0.1% to about 10%, preferably from about 1% to about 8%, and preferably from about 2.5% to about 7.5%, by weight, based on the total weight of the composition, including all ranges and subranges in between.

Suitable surfactants include, but are not limited to, surfactants having an HLB value of less than 8 and/or can include, for example, esters and ethers of sugars such as saccharose stearate, saccharose cocoate, sorbitan stearate, sobitan sesquioleate, and mixtures thereof; esters of fatty acids, in particular C8-C24, and preferably C16-C22, and polyols, in particular glycerol or sorbitol, such as glyceryl stearate, glyceryl laurate, polyglyceryl-2 stearate, PEG-100 stearate, sorbitan tristearate, and glyceryl ricinoleate; ethoxylated and/or propoxylated ethers such as the ethoxylated ether of stearyl alcohol with 2 ethylene oxide units (CTFA name “Steareth-2”); and a mixture of cyclomethicone/dimethicone copolyol such as that sold under the name Q2-3225C® by the company DOW CORNING.

Suitable desired agents include, for example, any pharmaceutically or cosmetically active agent, or any film forming agent known in the art. For example, a cosmetic makeup composition can comprise sufficient film forming agent to provide eyelashes with the desired film.

Suitable fibers include, but are not limited to, fibers which enable improvement of the lengthening effect. “Fiber” should be understood to mean an object of length L and diameter D such that L is much greater than D, D being the diameter of the circle in which the cross-section of the fibre is inscribed. In particular, the L/D ratio (or form factor) is selected in the band ranging from 3.5 to 2500, in particular from 5 to 500, and more particularly from 5 to 150. The fibers utilisable in the composition of the invention can be fibers of synthetic or natural origin, mineral or organic. They can be short or long, unitary or structured, for example, braided, hollow or full. They can be of any shape and in particular of circular or polygonal cross-section (square, hexagonal or octagonal) depending on the specific application envisaged. In particular, their ends are blunted and/or polished to avoid injury. They can be rigid or non-rigid fibers. They can be of synthetic or natural origin, mineral or organic. They can be surface treated or not, coated or not, and colored or not.

In one embodiment of the present invention, the compositions of the present invention are free of, substantially free of, or devoid of silicone oils.

Another preferred embodiment of the present invention is a composition which contains so little TEA-stearate that the presence of TEA-stearate does not affect the cosmetic properties of the composition. Preferably, the compositions are free of, substantially free of or devoid TEA-stearate.

According to other preferred embodiments, methods of treating, caring for and/or enhancing the appearance of eyelashes by applying compositions of the present invention to the eyelashes in an amount sufficient to treat, care for and/or enhance the appearance of the eyelashes are provided. In accordance with these preceding preferred embodiments, the compositions of the present invention are applied topically to the desired area of the eyelashes in an amount sufficient to treat, care for and/or enhance the appearance of the keratinous material. The compositions may be applied to the desired area as needed, preferably once or twice daily, more preferably once daily and then preferably allowed to dry before subjecting to contact such as with clothing or other objects (for example, a glass or a topcoat). Preferably, the composition is allowed to dry for about 1 minute or less, more preferably for about 45 seconds or less. The composition is preferably applied to the desired area that is dry or has been dried prior to application, or to which a basecoat has been previously applied.

According to preferred embodiments of the present invention, compositions having improved cosmetic properties such as, for example, improved waterproof, transfer-resistance, and/or long wear properties are provided.

According to preferred embodiments of the present invention, compositions having improved removability properties from eyelashes are provided. The improved removal properties result from the presence of more than 5% of the at least one sugar in the compositions. Preferably, compositions of the present invention have both improved cosmetic properties discussed in the preceding paragraph and improved removability properties. But it is also preferred that compositions of the present invention having improved removability properties have equivalent cosmetic properties discussed in the preceding paragraph as compared to identical compositions which lack at least one sugar—that is, it is also preferred that addition of more than at least 5% sugar does not substantially affect the cosmetic properties of the compositions.

According to preferred embodiments of the present invention, the compositions of the present invention can be removed using a water-containing composition which is essentially water (for example, greater than 50% water, at least 60% water, at least 70% water, at least 75% water, at least 80% water, at least 85% water, and at least 90% water), with the balance of the composition essentially being oil, although other ingredients may also be present in the compositions. For example, the composition comprising water can be a “topcoat” containing water which, when applied to a mascara composition of the present invention, facilitates removal of the mascara composition from the eyelashes. The amount of water to which the applied composition is exposed is sufficient to facilitate removal of the applied composition. Generally speaking, the amount of water applied to facilitate removal is preferably between 5 and 20 grams of water, preferably between 6 and 15 grams of water, and preferably between 7 and 10 grams of water. Although not wishing to be bound by any theory, it is believed that exposure to water causes the sugar and applied composition to break down, resulting in easier removal of the applied, dried composition from eyelashes, despite the fact that the composition is low-water-content and/or anhydrous.

According to preferred embodiments, the composition of the present invention and the water-containing composition (for example, topcoat) are present in a kit.

According to preferred embodiments of the present invention, methods of increasing removability of a low-water-content eyelash composition from eyelashes comprising adding at least one sugar to the composition in an amount sufficient to increase removability of the composition from eyelashes (after the comosition has been applied to eyelashes and allowed to dry) are provided. In accordance with these methods, the at least one sugar is added during preparation of the compositions of the present invention in the amounts discussed throughout this application.

According to preferred embodiments of the present invention, methods of removing an eyelash composition from eyelashes, wherein the eyelash composition comprises at least one sugar in an amount greater than 5% by weight of the eyelash composition and at least one oil and wherein the eyelash composition is low-water-content, comprising applying water to the eyelash composition in an amount sufficient to remove the eyelash composition from the eyelashes are provided.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention.

Notwithstanding that the numerical ranges and parameters setting forth the broad scope of the invention are approximations, the numerical values set forth in the specific examples are reported as precisely as possible. Any numerical value, however, inherently contain certain errors necessarily resulting from the standard deviation found in their respective measurements. The following examples are intended to illustrate the invention without limiting the scope as a result. The percentages are given on a weight basis.

### EXAMPLES

### Example 1—Sample Mascara Composition

| | | Amount by |
|---|---|---|
| Compound | Example | Weight |
| Volatile Oil | Isododecane | 35-60% |
| Waxes | Candelilla, Carnauba, | 10-25% |
| | Beeswax, Rice Bran | |
| | Wax | |
| Clay | Hectorite | 3-10% |
| Film Forming Agents | | 5-15% |
| Coloring Agents/Fillers | Pigments | 5-20% |
| Sugar | Sucrose | More than 5% |
| Solvents/Antioxidants/Solvents | | 1-10% |
| Other Cosmetically- | | |
| Acceptable Ingredients | | |

### Example 2—Removability Testing

Compositions

Mascara Formulations

Identical invention and comparative compositions were prepared based on the formulation from example 1. The only differences in the compositions were the amount of isododecane present (QS), and whether sugar or salt was present.

Comparative composition 1 contained 0% sugar and 0% salt.

Comparative composition 2 contained 10% calcium chloride.

Comparative composition 3 contained 10% sodium chloride.

Invention composition 1 contained 5% sucrose.

Invention composition 2 contained 10% sucrose.

Removing Solutions

Two commercially-available eyelash composition removers were used. Commercial remover 1 contained 60% water. Commercial remover 2 contained 85% water.

Protocol

Invention and comparative mascara formulations were casted into a film on a drawdown paper using 1 ml drawdown bar, and air dried under room temperature overnight. Each removing solution was placed 3 drops onto the dried mascara film, and 10 s, 30 s and 60 s respectively were allowed to pass for each drop to allow time to soak. A kimwipe was folded twice for removing. Each removing solution drop was wiped 5 times and the remaining film was checked. The removability was rated based on residue of mascara on the drawdown card, from 0 (not removed)-5 (most removed)

Results

| | Commercial Remover 1 | | | Commercial Remover 2 | | |
|---|---|---|---|---|---|---|
| | (60% water) | | | (85% water) | | |
| | 10 sec | 30 sec | 60 sec | 10 sec | 30 sec | 60 sec |
| Comparative 1 | 1 | 0 | 0 | 0 | 0 | 0.5 |
| Invention 1 | 0 | 0.75 | 0.75 | 0 | 0.5 | 0.25 |
| with 5% | | | | | | |
| sucrose | | | | | | |
| Invention 2 | 1 | 4.5 | 4.5 | 0.75 | 3.5 | 4 |
| with 10% | | | | | | |
| sucrose | | | | | | |
| Comparative 1 | 0 | 0 | 0 | 0 | 0 | 0 |
| Comparative 2 | 0 | 0 | 0 | 0 | 0 | 0 |
| with | | | | | | |
| 10% CaC12 | | | | | | |
| Comparative 3 | 0 | 0 | 0 | 0 | 0 | 0 |
| with | | | | | | |
| 10% NaCl | | | | | | |

What is claimed is:

1. An eyelash composition comprising at least one sugar in an amount greater than 5% by weight of the eyelash composition and at least one oil, wherein the eyelash composition is low-water-content.

2. The eyelash composition of claim 1, wherein the at least one oil is a volatile oil.

3. The eyelash composition of claim 1, further comprising at least one wax.

4. The eyelash composition of claim 1, further comprising at least one coloring agent.

5. The eyelash composition of claim 1, in the form of a mascara.

6. The eyelash composition of claim 1, wherein the at least one oil and the at least one sugar are present in the eyelash composition in a weight ratio of from 10:1 to 1.1:1.

7. The eyelash composition of claim 1, wherein the sugar is sucrose.

8. The eyelash composition of claim 1, wherein the sugar is present in an amount of at least 10% by weight of the eyelash composition.

9. The eyelash composition of claim 1, wherein the sugar is present in an amount of at least 15% by weight of the eyelash composition.

10. The eyelash composition of claim 1, wherein the sugar is present in an amount of at least 20% by weight of the eyelash composition.

11. A method of removing an eyelash composition from eyelashes, wherein the eyelash composition comprises at least one sugar in an amount greater than 5% by weight of the eyelash composition and at least one oil and wherein the eyelash composition is low-water-content, comprising applying water to the eyelash composition in an amount sufficient to remove the eyelash composition from the eyelashes.

12. A method of increasing removability of an eyelash composition from eyelashes, wherein the eyelash composition is low-water-content, comprising adding during preparation of the eyelash composition at least one sugar in an amount greater than 5% by weight of the eyelash composition to the eyelash composition, resulting in increased removability of the eyelash composition from eyelashes.

13. The eyelash composition of claim 1, wherein the eyelash composition is a mascara composition comprising:

| Compound | Amount by Weight |
|---|---|
| Volatile Oil | 35-60% |
| Waxes | 10-25% |
| Clay | 3-10% |
| Film Forming Agents | 5-15% |
| Coloring Agents/Fillers | 5-20% |
| Sugar | More than 5% |
| Solvents/Antioxidants/Solvents | 1-10% |
| Other Cosmetically- | |
| Acceptable Ingredients. | |
